(19) United States
(12) Patent Application Publication
(10) Pub. No.: US 2025/0368940 A1
(43) Pub. Date: Dec. 4, 2025

(54) DEVICE FOR CELL SEEDING AND CELL SEEDING METHOD

(71) Applicants: CANON MEDICAL SYSTEMS CORPORATION, Otawara-shi (JP); Canon Kabushiki Kaisha, Tokyo (JP)

(72) Inventors: Kenta KAMINO, Shimotsuga (JP); Shinya SUGIYAMA, Nasushiobara (JP)

(73) Assignees: CANON MEDICAL SYSTEMS CORPORATION, Otawara-shi (JP); Canon Kabushiki Kaisha, Tokyo (JP)

(21) Appl. No.: 19/224,967

(22) Filed: Jun. 2, 2025

(30) Foreign Application Priority Data
Jun. 4, 2024 (JP) 2024-090953

(51) Int. Cl. C12M 1/26; C12M 1/00

(52) U.S. Cl. CPC C12M 33/00; C12M 23/50

(57) ABSTRACT

A device for cell seeding according to an exemplary embodiment includes a first layer and a second layer. The first layer includes a first accommodation space configured to accommodate a cell to be seeded. The second layer includes a first through-hole wider than the cell and is positioned vertically below the first layer. Then, the device for cell seeding forms a first state in which the first through-hole is positioned at a bottom surface of the first accommodation space by changing a relative position between the first layer and the second layer in a horizontal direction.

### CROSS-REFERENCE TO RELATED APPLICATIONS

This application is based upon and claims the benefit of priority from Japanese Patent Application No. 2024-090953, filed Jun. 4, 2024, the entire contents of which are incorporated herein by reference.

### FIELD

Embodiments described herein relate generally to a device for cell seeding and a cell seeding method.

### BACKGROUND

Conventionally, cells are cultured on a culture dish. A technician may pick up cells from a specific cell colony cultured on the culture dish.

However, in a case where the cells are placed randomly on the culture dish, the cell colonies cultured on the culture dish may overlap. In this case, the technician can pick up a cell from an unintended colony due to the overlapping colonies. Thus, a device capable of individually seeding cells is in demand.

### BRIEF DESCRIPTION OF THE DRAWINGS

FIG. 1 is a diagram illustrating an example of a configuration of a culture kit according to a first exemplary embodiment;

FIG. 2 is a cross-sectional view illustrating an example of a cross-section of the culture kit according to the first exemplary embodiment;

FIGS. 3A to 3C are top views illustrating states of a device for cell seeding according to the first exemplary embodiment;

FIG. 4 is a diagram illustrating an example of the culture kit in a case where a cell suspension is dispensed dropwise;

FIG. 5 is a diagram illustrating an example of the culture kit in a case where cells are dropped;

FIG. 6 is a diagram illustrating an example of the culture kit in a case where cells are placed in a stationary manner;

FIG. 7 is a diagram illustrating an example of the culture kit in a case where a cell suspension is dispensed dropwise;

FIG. 8 is a diagram illustrating an example of the culture kit in a case where cells are isolated;

FIG. 9 is a diagram illustrating an example of the culture kit in a case where cells are dropped; and

FIG. 10 is a diagram illustrating an example of the culture kit in a case where cells are placed in a stationary manner.

### DETAILED DESCRIPTION

A device for cell seeding according to an exemplary embodiment includes a first layer and a second layer. The first layer includes a first accommodation space configured to accommodate a cell to be seeded. The second layer includes a first through-hole wider than the cell and is positioned vertically below the first layer. Then, the device for cell seeding forms a first state in which the first through-hole is positioned at a bottom surface of the first accommodation space by changing a relative position between the first layer and the second layer in a horizontal direction.

Various Embodiments will be described hereinafter with reference to the accompanying drawings.

In the exemplary embodiments described below, portions that perform similar operations are assigned the same reference numeral, and redundant descriptions are omitted as appropriate.

### First Exemplary Embodiment

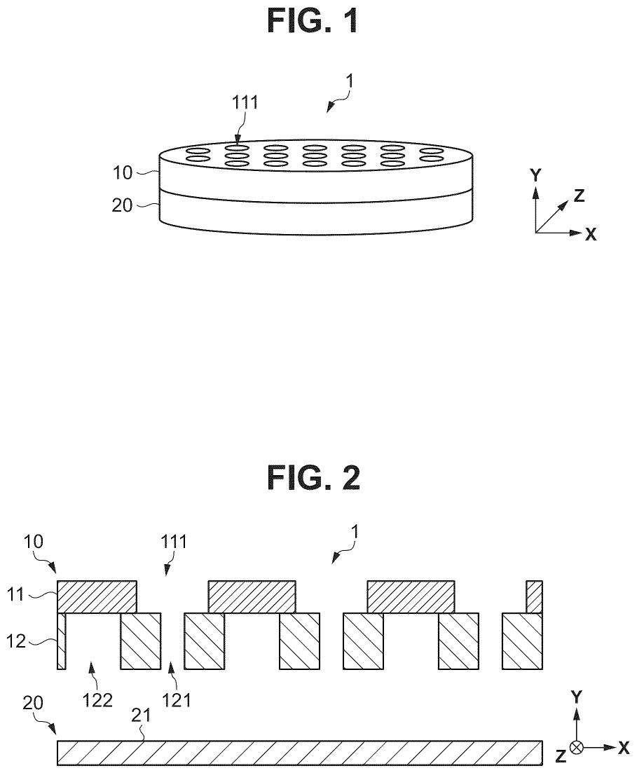

FIG. 1 is a diagram illustrating an example of a configuration of a culture kit 1 according to a first exemplary embodiment. The culture kit 1 includes a device 10 for cell seeding and a culture dish 20. The device 10 for cell seeding is equipment for seeding cells individually. The culture dish 20 is equipment for culturing the cells seeded by the device 10 for cell seeding. The device 10 for cell seeding is positioned vertically above the culture dish 20. Then, the culture dish 20 cultures the cells dropped from the device 10 for cell seeding.

The device 10 for cell seeding and the culture dish 20 may be integrated or independent of each other. Furthermore, the device 10 for cell seeding illustrated in FIG. 1 has approximately the same shape as that of the culture dish 20. However, the device 10 for cell seeding may have a different shape from the culture dish 20. Furthermore, the device 10 for cell seeding is not limited to a circular shape and may be rectangular or may have another shape.

FIG. 2 is a cross-sectional view illustrating an example of a cross-section of the culture kit 1 according to the first exemplary embodiment. The device 10 for cell seeding includes an accommodation layer 11 and a passage layer 12.

The accommodation layer 11 includes a plurality of accommodation portions 111 capable of accommodating cells Cl to be seeded. The accommodation layer 11 is an example of a first layer. The accommodation layer 11 is a plate-shaped component. The accommodation layer 11 is superposed on an upper surface of the passage layer 12.

The accommodation portions 111 are holes formed through the accommodation layer 11. In other words, the accommodation portions 111 are holes extending to the passage layer 12. A cell suspension is dispensed dropwise into the accommodation portions 111. Then, the accommodation portions 111 accommodate cells C1 contained in the cell suspension. Each accommodation portion 111 is a space with sufficient depth and width to accommodate a single cell C1. The size of each accommodation portion 111 may be changed depending on a target to be accommodated. For example, in the case of accommodating peripheral blood mononuclear cells (PBMCs), each accommodation portion 111 may have a size of approximately 10 um in a horizontal direction.

The passage layer 12 includes drop openings 122 and is positioned vertically below the accommodation layer 11. Each drop opening 122 is wider than the cell C1. The passage layer 12 is an example of a second layer. The drop opening 122 is an example of a first through-hole. The passage layer 12 is a plate-shaped component. Further, on the passage layer 12, a lower surface of the accommodation layer 11 is superposed. Further, the passage layer 12 includes a plurality of combinations of the drop openings 122 and liquid discharge openings 121. Each liquid discharge opening 121 is narrower than the cell C1. The liquid discharge opening 121 is an example of a second through-hole.

The liquid discharge openings 121 are holes formed through the passage layer 12. Further, the liquid discharge openings 121 are formed to be narrower than the cells C1 contained in the cell suspension in the horizontal direction. In other words, the diameter of the liquid discharge openings 121 in the horizontal direction is smaller than the diameter of the cells C1. Thus, the cells C1 contained in the cell suspension cannot pass through the liquid discharge openings 121. On the other hand, liquid contained in the cell suspension can pass through the liquid discharge openings 121.

The drop openings 122 are holes formed through the passage layer 12. In other words, the drop openings 122 are holes extending to the culture dish 20. Further, the drop openings 122 are formed to be wider than the cells C1 contained in the cell suspension. In other words, the diameter of the drop openings 122 in the horizontal direction is larger than the diameter of the cells C1. Thus, the cells C1 contained in the cell suspension pass through the drop openings 122 and are dropped onto the culture dish 20.

The culture dish 20 has a cell seeding surface 21 on its upper surface. The cell seeding surface 21 is a surface provided with a culture medium for culturing the cells C1. The cell seeding surface 21 cultures the adherent cells C1.

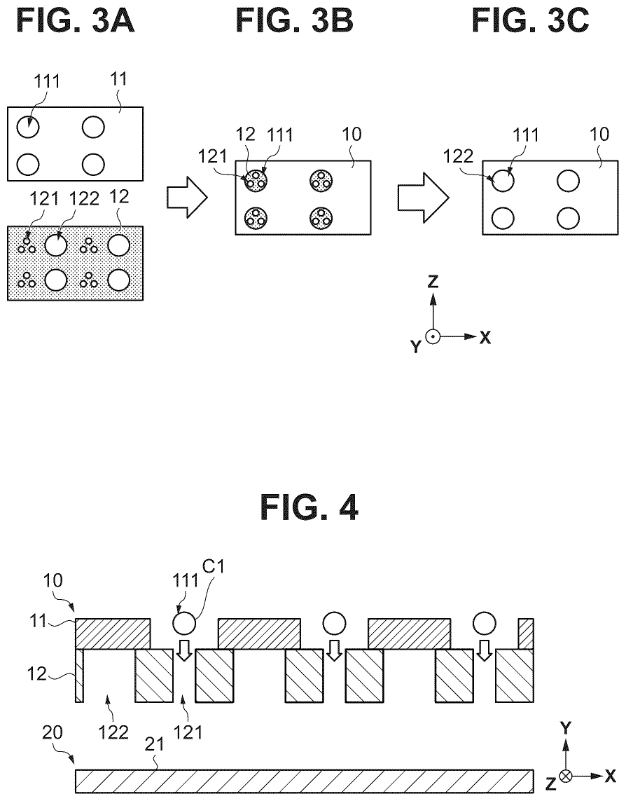

FIGS. 3A to 3C are top views illustrating states of the device 10 for cell seeding according to the first exemplary embodiment. FIG. 3A is a top view illustrating a state where the accommodation layer 11 and the passage layer 12 are not superposed. FIG. 3B is a top view illustrating a liquid-discharge-state arrangement. FIG. 3C is a top view illustrating a dropping-state arrangement.

The device 10 for cell seeding has an accommodation-state arrangement and the dropping-state arrangement based on the arrangement of the accommodation layer 11 and the passage layer 12. As illustrated in FIG. 3A, the accommodation layer 11 includes the plurality of accommodation portions 111. The passage layer 12 includes a combination of the liquid discharge openings 121 and the drop opening 122 for each accommodation portion 111.

In FIG. 3A, three liquid discharge openings 121 are provided for each accommodation portion 111. However, one liquid discharge opening 121 may be provided for each accommodation portion 111, or two or four or more liquid discharge openings 121 may be provided for each accommodation portion 111. Furthermore, the passage layer 12 may not include any liquid discharge openings 121.

The liquid discharge openings 121 and the drop openings 122 are arranged next to each other. With this arrangement, a technician can switch between the liquid-discharge-state arrangement and the dropping-state arrangement by sliding the accommodation layer 11 or the passage layer 12. For example, the technician slides the accommodation layer 11. In other words, the device 10 for cell seeding switches between the accommodation-state arrangement, in which each liquid discharge opening 121 is positioned on a bottom surface of the accommodation portion 111, and the dropping-state arrangement by changing a relative position between the accommodation layer 11 and the passage layer 12 in the horizontal direction. The accommodation-state arrangement is an example of a second state.

The accommodation-state arrangement is an arrangement that is used to accommodate the cells C1 contained in the cell suspension dispensed dropwise into the accommodation portions 111. In other words, as illustrated in FIG. 3B, the liquid discharge openings 121 are positioned at the bottom surfaces of the accommodation portions 111 of the accommodation layer 11 in the accommodation-state arrangement. The accommodation-state arrangement is an arrangement that, in a case where the cell suspension is dispensed dropwise into the accommodation portions 111, discharges the liquid through the liquid discharge openings 121 and retains the cells C1 in the accommodation portions 111. Further, the cells C1 move toward the bottom surface of the accommodation portion 111 due to a flow of the liquid discharged through the liquid discharge openings 121. Thus, presence of the liquid discharge openings 121 facilitates accommodation of the cells C1 in the accommodation portions 111 by the technician.

The device 10 for cell seeding forms the dropping-state arrangement, in which each drop opening 122 is positioned at the bottom surface of the accommodation portion 111, by changing the relative position between the accommodation layer 11 and the passage layer 12 in the horizontal direction. The dropping-state arrangement is an arrangement that is used to drop the cells C1 accommodated in the accommodation portions 111 onto the culture dish 20. In other words, as illustrated in FIG. 3C, the drop openings 122 are positioned at the bottom surfaces of the accommodation portions 111 of the accommodation layer 11 in the dropping-state arrangement. This allows the cells C1 retained in the accommodation portions 111 to be dropped through the drop openings 122 in the dropping-state arrangement.

A distance between the center of the accommodation portion 111 and the drop opening 122 and the center of another accommodation portion 111 and another drop opening 122 in the dropping-state arrangement corresponds to a distance between the cells C1 placed in a stationary manner on the cell seeding surface 21. Further, the distance between the cells C1 placed in a stationary manner on the cell seeding surface 21 corresponds to a distance between colonies of the cells C1 cultured on the cell seeding surface 21. Thus, the distance between the center of the accommodation portion 111 and the drop opening 122 and the center of the other accommodation portion 111 and the other drop opening 122 in the dropping-state arrangement is determined based on a distance required for the colonies. For example, the distance between the center of the accommodation portion 111 and the drop opening 122 and the center of the other accommodation portion 111 and the other drop opening 122 in the dropping-state arrangement is 200 um to 2000 um. Further, the accommodation portions 111 and the drop openings 122 illustrated in FIGS. 3A to 3C are arranged in parallel. However, the accommodation portions 111 and the drop openings 122 may be arranged in a close-packed structure.

The device 10 for cell seeding may include a drive unit (not illustrated) configured to change the relative position between the accommodation layer 11 and the passage layer 12. The drive unit supplies power to slide the accommodation layer 11 or the passage layer 12. For example, the drive unit slides the accommodation layer 11 or the passage layer 12 by 100 μm to 1000 μm.

For example, the drive unit is a unit configured to slide the passage layer 12 using magnetic force. More specifically, the drive unit moves the passage layer 12 by supplying the magnetic force to a magnetic material provided to a portion of the passage layer 12. The drive unit is not limited to a unit that slides the passage layer 12 using the magnetic force, and may also be a unit that slides the passage layer 12 using power of a motor, an actuator, a solenoid valve, or a mechanism that slides the passage layer 12 using a different form of power.

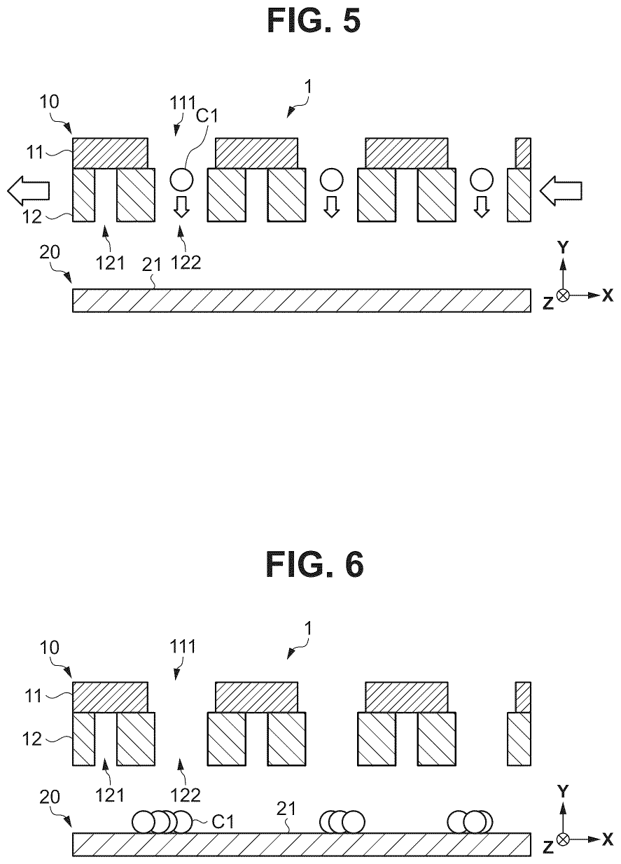

Next, an operating procedure for the device 10 for cell seeding according to the first exemplary embodiment will be described. FIG. 4 is a diagram illustrating an example of the culture kit 1 in a case where the cell suspension is dispensed dropwise. FIG. 5 is a diagram illustrating an example of the culture kit 1 in a case where the cells C1 are dropped. FIG. 6 is a diagram illustrating an example of the culture kit 1 in a case where the cells C1 is placed in a stationary manner.

In the accommodation-state arrangement illustrated in FIG. 4, the technician dispenses the cell suspension dropwise into the accommodation portions 111 of the accommodation layer 11. In the case of the accommodation-state arrangement, each liquid discharge opening 121 is positioned at the bottom surface of the accommodation portion 111. The diameter of the liquid discharge openings 121 is smaller than the diameter of the cells C1. Thus, the liquid discharge openings 121 allow the liquid to pass therethrough but not the cells C1. As a result, the accommodation portions 111 accommodate the cells C1 contained in the cell suspension dispensed dropwise.

The technician slides the passage layer 12 using the drive unit. As a result, the device 10 for cell seeding is switched to the dropping-state arrangement illustrated in FIG. 5. The diameter of the drop openings 122 is larger than the diameter of the cells C1. Thus, the cells C1 drop through the drop openings 122.

After dropping through the drop openings 122, the cells C1 are place in a stationary manner on the cell seeding surface 21. Then, the cells C1 are cultured on the cell seeding surface 21. Specifically, colonies including a plurality of cells C1 are formed on the cell seeding surface 21 as illustrated in FIG. 6.

As described above, the device 10 for cell seeding according to the first exemplary embodiment includes the accommodation layer 11 and the passage layer 12. The accommodation layer 11 includes the accommodation portions 111 configured to accommodate the cells C1, and the passage layer 12 includes the drop openings 122 configured to allow the cells C1 to pass through. Then, the device 10 for cell seeding positions the drop openings 122 at the bottom surface of the accommodation portion 111 by changing the relative position between the accommodation layer 11 and the passage layer 12 in the horizontal direction. As described above, the device 10 for cell seeding can seed the cells C1 individually.

The cells C1 accommodated in the accommodation portions 111 pass through the drop openings 122 and are place in a stationary manner on the cell seeding surface 21. Then, the culture dish 20 generates colonies individually by culturing each cell C1 on the cell seeding surface 21. In a case where a distance between the accommodation portion 111 and the drop opening 122 and an adjacent accommodation portion 111 and an adjacent drop opening 122 is greater than the size of the generated colonies, the device 10 for cell seeding can prevent the colonies from overlapping.

### First Modification

There is a case where the accommodation portions 111 of the accommodation layer 11 accommodate the plurality of cells C1 stacked in a depth direction, i.e., Y-axis direction. In this case, when the device 10 for cell seeding is switched to the dropping-state arrangement, the device 10 for cell seeding drops the plurality of stacked cells C1 onto the cell seeding surface 21. Then, a colony is formed based on the plurality of cells C1 on the cell seeding surface 21.

Thus, a device 10a for cell seeding according to the first modification prevents the plurality of stacked cells C1 from dropping onto the cell seeding surface 21. As a result, a colony is formed based on the individual cell C1 on the cell seeding surface 21.

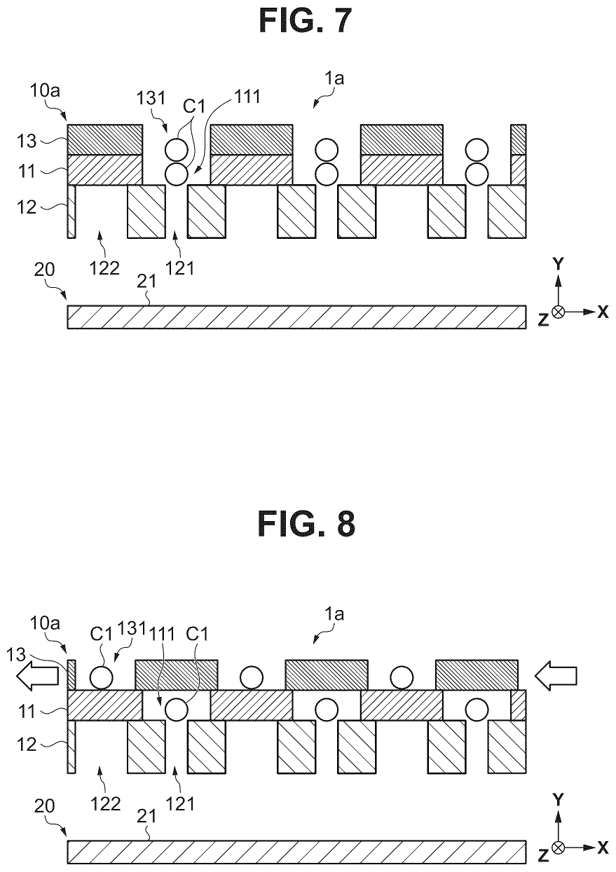

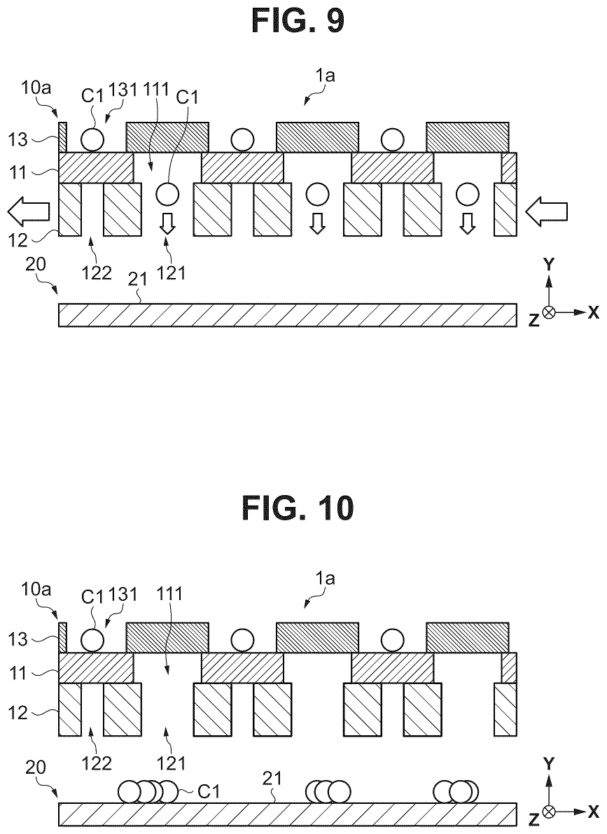

FIG. 7 is a diagram illustrating an example of a culture kit la in a case where the cell suspension is dispensed dropwise. FIG. 8 is a diagram illustrating an example of the culture kit la in a case where the cells Cl are isolated. FIG. 9 is a diagram illustrating an example of the culture kit la in a case where the cells Cl are dropped. FIG. 10 is a diagram illustrating an example of the culture kit la in a case where the cells C1 are placed in a stationary manner.

The device 10a for cell seeding includes an isolation layer 13, the accommodation layer 11, and the passage layer 12.

The isolation layer 13 is a layer configured to isolate the plurality of cells C1 stacked in the depth direction. The isolation layer 13 includes isolation portions 131 and is positioned vertically above the accommodation layer 11. The width of the isolation portions 131 is approximately the same as the width of the accommodation portions 111. The isolation layer 13 is an example of a third layer. The isolation portions 131 are an example of a second accommodation space. More specifically, the isolation layer 13 is a plate-shaped component. Further, the isolation layer 13 is superposed on an upper surface of the accommodation layer 11. The isolation layer 13 includes the isolation portions 131 configured to isolate the plurality of cells C1 stacked in the depth direction in a case where the isolation layer 13 is slid. The isolation portions 131 are holes formed through the isolation layer 13. In other words, the isolation portions 131 are holes extending to the accommodation layer 11.

The accommodation layer 11 includes the accommodation portions 111 configured to accommodate a single cell C1 in the depth direction. The accommodation layer 11 is a plate-shaped component. Further, on the accommodation layer 11, a lower surface of the isolation layer 13 is superposed. The accommodation portions 111 are holes formed through the accommodation layer 11. In other words, the accommodation portions 111 are holes extending to the passage layer 12. Further, the height of the accommodation portions 111 in the depth direction, i.e., Y-axis direction is approximately the same as the height of the cells C1.

The device 10a for cell seeding has the accommodation-state arrangement and an isolation-state arrangement based on the arrangement of the isolation layer 13 and the accommodation layer 11. The accommodation-state arrangement is an arrangement in which the isolation portions 131, the accommodation portions 111, and the liquid discharge openings 121 are arranged to overlap in the depth direction as illustrated in FIG. 7. The accommodation-state arrangement is used to accommodate the cells C1 contained in the cell suspension dispensed dropwise into the accommodation portions 111.

The isolation-state arrangement changes the relative position between the accommodation portions 111 and the isolation portions 131 in the horizontal direction by changing the relative position between the isolation layer 13 and the accommodation layer 11 in the horizontal direction. As a result, the cells C1 stacked in the depth direction and accommodated in a space formed by the isolation layer 13 and the accommodation layer 11 are isolated in the isolation-state arrangement. In other words, the isolation-state arrangement is an arrangement in which the isolation layer 13 or the accommodation layer 11 is slid from the accommodation-state arrangement as illustrated in FIG. 8. The height of the accommodation portions 111 in the depth direction, i.e., Y-axis direction, is approximately the same as the height of the cells C1. Thus, a single cell C1 remains in each accommodation portion 111. In other words, the device 10a for cell seeding can isolate the cells C1.

Next, an operating procedure for the device 10a for cell seeding according to the first modification will be described.

In the accommodation-state arrangement illustrated in FIG. 7, the technician dispenses the cell suspension dropwise into accommodation spaces formed by the isolation portions 131 and the accommodation portions 111.

In the case of the accommodation-state arrangement, the accommodation portions 111 and the isolation portions 131 are arranged to overlap. Further, the liquid discharge openings 121 are positioned at the bottom surfaces of the accommodation portions 111. The diameter of the liquid discharge openings 121 is smaller than the diameter of the cells C1. Thus, the liquid discharge openings 121 allow the liquid to pass therethrough but not the cells C1. As a result, the isolation portions 131 and the accommodation portions 111 accommodate the cells C1 contained in the cell suspension dispensed dropwise.

The technician slides the accommodation layer 11 as illustrated in FIG. 8. The height direction, i. e., Y-axis direction, of the accommodation portions 111 is substantially the same as the height of the cells C1. Thus, the device 10a for cell seeding can isolate the plurality of cells C1 stacked in the depth direction.

The technician slides the passage layer 12. As a result, the device 10a for cell seeding is switched to the dropping-state arrangement illustrated in FIG. 9. The diameter of the drop openings 122 is larger than the diameter of the cells C1. Thus, the cells C1 drop through the drop openings 122.

After dropping through the drop openings 122, the cells C1 are place in a stationary manner on the cell seeding surface 21. Then, the cells C1 are cultured on the cell seeding surface 21. Specifically, colonies including a plurality of cells Cl are formed on the cell seeding surface 21 as illustrated in FIG. 10.

The technician may slide the accommodation layer 11 in the accommodation-state arrangement illustrated in FIG. 7. In a case where the accommodation layer 11 is slid, the device 10a for cell seeding is switched to the dropping-state arrangement illustrated in FIG. 9. With this arrangement, the device 10a for cell seeding may drop the cells C1 through the drop openings 122.

As described above, in a case where the plurality of cells C1 stacked in the depth direction is accommodated in the spaces formed by the accommodation portions 111 and the isolation portions 131, the device 10a for cell seeding according to the first modification can isolate the plurality of cells Cl stacked in the depth direction by moving the relative position between the accommodation layer 11 and the isolation layer 13 in the horizontal direction. In other words, a single cell C1 is accommodated in each accommodation portion 111. Thus, the device 10a for cell seeding can prevent the plurality of cells C1 stacked in the depth direction from being seeded in this state.

At least one exemplary embodiment described above can provide a device capable of individually seeding the cells C1.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A device for cell seeding, the device comprising:
a first layer including a first accommodation space configured to accommodate a cell to be seeded; and
a second layer including a first through-hole wider than the cell and positioned vertically below the first layer,
wherein a first state in which the first through-hole is positioned at a bottom surface of the first accommodation space is formed by changing a relative position between the first layer and the second layer in a horizontal direction.

2. The device for cell seeding according to claim 1,
wherein the second layer includes the first through-hole and a second through-hole narrower than the cell, and
wherein a second state in which the second through-hole is positioned at the bottom surface of the first accommodation space and the first state are switched by changing the relative position between the first layer and the second layer in the horizontal direction.

3. The device for cell seeding according to claim 2,
wherein the first state is an arrangement that discharges a liquid through the second through-hole and retains the cell in a case where a cell suspension is dispensed dropwise into the first accommodation space, and
wherein the second state is an arrangement that drops the cell retained in the first accommodation space through the second through-hole.

4. The device for cell seeding according to claim 1, further comprising a drive unit configured to change the relative position between the first layer and the second layer.

5. The device for cell seeding according to claim 1, further comprising a third layer including a second accommodation space with approximately a same width as a width of the first accommodation space and positioned vertically above the first layer,
wherein the first accommodation space accommodates a single cell in a depth direction,
wherein in the first state, the first accommodation space and the second accommodation space are positioned to overlap, and
wherein a relative position between the first accommodation space and the second accommodation space is changed in the horizontal direction by changing the relative position between the third layer and the first layer in the horizontal direction.

6. A cell seeding method comprising causing a device for cell seeding to form a first state in which a first through-hole is positioned at a bottom surface of a first accommodation space by changing a relative position between a first layer and a second layer in a horizontal direction,
wherein the device includes:
the first layer including the first accommodation space configured to accommodate a cell to be seeded; and
the second layer including the first through-hole wider than the cell and positioned vertically below the first layer.
